(19) United States
(12) Patent Application Publication
(10) Pub. No.: US 2023/0278880 A1
(43) Pub. Date: Sep. 7, 2023

(54) Ammonium Bicarbonate Production Method Using Anaerobic Digester Outputs

(71) Applicant: Sustain Technologies, LLC, Charlotte, NC (US)

(72) Inventor: Robert Hickey, Okemos, MI (US)

(21) Appl. No.: 17/688,015

(22) Filed: Mar. 7, 2022

(51) Int. Cl. C01C 1/26; C12N 1/20; C02F 9/00; B01D 19/00; B01D 53/14

(52) U.S. Cl. CPC C01C 1/26; C12N 1/20; C02F 9/00; B01D 19/0005; B01D 53/1493; B01D 53/1418; B01D 53/1425; C02F 3/28

(57) ABSTRACT

A method of recovering ammonia bicarbonate from a biogas and digestate liquor uses an arrangement of equipment in an operational arrangement that requires relatively modest capital investment and that operates with simplicity to provide solid ammonia bicarbonate. In a specific form the method achieves this by upgrading of biogas to produce a rejected stream having a high mole fraction of CO₂ at an elevated pressure that eliminates the need for further compression when using the CO₂ for recovery of ammonia bicarbonate. The method operates can operate at a high pH produced inherently in a digestate stripper by concurrently stripping dissolved CO₂ from solution to improve the simplicity of solid AB recovery.

### FIELD OF THE INVENTION

The present invention relates to methods for recovering ammonium bicarbonate from organic waste streams (aka biowaste) and producing renewable natural gas (RNG). More specifically the present invention relates to recovering ammonium bicarbonate from an ammonia rich manure and/or anaerobic digestate using CO₂ off gas from the purification of the raw biogas to provide an ammonium containing slurry or solution that contains ammonium bicarbonate (AB) as one of the principal ammonium containing compounds.

### BACKGROUND OF THE INVENTION

The digestion of many organic materials that contain nitrogen compounds results in the production of ammonia and ammonium compounds recoverable as an ammonium product that has high value.

There are many process and flow arrangements in combination with inorganic chemical additives to recover ammonia and ammonium compounds such as ammonium salts from wastewater. Such processes commonly strip ammonia from wastewater streams by elevating the pH of the wastewater and then recovering the ammonia with an acidic scrubbing solution such as sulfuric acid, hydrochloric acid or nitric acid. Stripping towers, steam strippers, vacuum strippers, membrane systems, RO systems and crystallizers in various combinations have been used or proposed for use to recover ammonia from wastewater.

Methods are thus sought to recover renewable or green AB from wastewater streams, especially wastewater generated in processing of organic matter. Especially suitable waste sources for AB production include farm waste from animals and in particular manure. AB and other ammonia-based compounds from such sources can provide organic AB that also satisfy feed origin requirements for the supply of green ammonia and ammonia compounds.

AB is a particularly desirable compound derivable from the by products in the production of biogas from biological waste materials. AB finds a wide variety of uses from agriculture to baking. It often supplies the nitrogen for fertilizers.

The anaerobic digestion produces a digestate stream and a biogas. Biogas from anaerobic digestion is usually laden with CO₂ and purification of the biogas to useable methane, typically in the form of a RNG stream, also provides a gas stream rich in CO₂. The digestate and the biogas may contain a variety of other chemical compounds in addition to ammonia compounds. These compounds regularly include sulfur compounds most prevalently as H2S. Treatment of the biogas by oxidation of the H2S can provide a source of sulfuric acid.

When recovering AB, nitrogen or other nitrogenous compounds, digestate in the form of a liquor and/or a sludge typically undergoes stripping followed by scrubbing and/or adsorption that yields an effluent stream containing recoverable nitrogenous compounds such as AB. Contact of the effluent from the stripping step in a scrubber and with a CO₂ containing stream produces recoverable AB dissolved in the liquid effluent from a stripping gas scrubber. Increasing the pH of the digestate liquor, usually in a stripping step, moves the equilibrium of the ammonia away from dissolved ionized ammonia and toward ammonia gas. In most cases the addition of chemicals achieves this shift.

Further, in the recovery of ammonia and ammonium compounds, U.S. Pat. No. 7,811,455 (Burke) contacts the effluent from a digestate stripper with CO₂ from biogas purification to produce AB from the ammonia generated in the stripper. Burke then precipitates AB for eventual recovery as an AB product. Burke again uses chemicals to increase the pH in the stripper to increase the ammonia emanating therefrom. Thus, the Burke method disadvantageously produces non-organic AB i.e., not marketable as green ammonia products.

U.S. Pat. No. 8,486,359 (Hickey); U.S. Pat. No. 8,580,219 (Hickey); U.S. Pat. No. 10,793,458 (Orentlicher, et al.); U.S. Pat. No. 10,604,432 (Bassani et. al.) and U.S. Pat. No. 10,106,447 (Orentlicher, et al.) disclose methods for recovering ammonium carbonate (AC), AB, ammonia, and ammonia-based compounds from wastewater associated with the digestion of organic wastes.

More specifically U.S. Pat. Nos. 8,486,359 and 8,850,219 disclose methods, and systems for recovering and concentrating dissolved AB and AC from a liquid solution comprising ammonia or ammonia compounds. A specific liquid solution is an anaerobic digestion liquor. The method strips the liquid solution with a gas, in many cases air, to produce a gas containing ammonia that passes to a scrubber where contact with a CO₂ rich gas produces ammonium compounds such as AB and AC.

U.S. Pat. Nos. 10,793,458, 10,604,432, and 10,106,447 show recovery of AC and AB by anaerobically digesting agricultural, municipal and/or industrial wastewater and concentrating gaseous ammonia to produce AB from a wastewater containing ammonia using gas separation, condensation, and filtration, at controlled operating temperatures. Some of the methods disclosed therein use crystallization to concentrate the AB. The method is carried out without the use of chemicals. The method teaches starting with wastewater at temperature of at least 80° C.; concentrating dissolved AC and AB using reverse osmosis at a temperature of between about 35 and 50° C.; and crystallizing concentrated dissolved AC and AB at a temperature of less than about 35° C. to form solid AB and AC.

Thus, in the recovery of ammonium compounds, and in particular a high recovery of AB, the prior art puts forth a multitude of processing equipment in many different arrangements as well as wide range of operational parameters with varying ranges. The availability and variability of these factors presents a substantial challenge in determining which equipment arrangement and operational conditions will yield a highly efficient and cost effect recovery of AB. Also, for the purpose of cost reduction, methods that produce AB with a minimum of equipment will provide substantial benefit. As a result, the challenge of improving the recovery of AB and reduced cost and higher efficiency goes well beyond simple optimization of selected parameters and equipment but requires a discovery effort and unique insight.

As a limited example the prior art discloses that higher temperatures when stripping ammonia from a digestate stripper will yield higher stripping efficiency and higher overall ammonia recovery. In addition, it is known that higher temperatures when contacting ammonia with CO₂ increases the solubility of AB in a recovered AB solution, but lower temperatures when contacting ammonia with CO₂ will dissolve more CO₂ and also result in higher dissolved CO₂ levels that can also provide more efficiency in the AB production. Finally, the particular equipment chosen to effect such contacting and its arrangement presents multiple layers of complexity to improving the production of AB from digestate streams. Moreover, the above description of the processes considers the effects of only a few of operational parameters of the many that need consideration in the obtaining meaningful improvement in such methods.

Thus, there is a need to find new methods for the efficient generation of ammonia compounds and renewable natural gas from waste streams to produce ammonia and particularly ammonia-based compounds such as AB an AC. In particular, new and highly flexible methods are needed to adapt to the varieties of available renewable waste and especially animal waste from agricultural operations. Methods that can adapt to a wide range of agricultural sources provide especially useful capability to use renewable sources that thereby provide green ammonia/ammonia compounds and green natural gas.

### SUMMARY OF THE INVENTION

This invention provides a specific arrangement of equipment in an operational method that uses relatively modest capital investment and that operates with simplicity to provide AB by processing a biogas and a digestate liquor from an anaerobic digester that receives a biological wastewater stream. In one respect the method achieves this by upgrading of biogas to produce a rejected stream having a high mole fraction of CO₂ at a high enough pressure to eliminate the need for further compression when using the CO₂ for recovery of AB. In another respect the process operates at a high pH produced inherently in the digestate stripper by concurrently stripping dissolved CO₂ from solution to improve the simplicity of AB recovery.

Briefly, in the method disclosed herein a manure or digestate stripper contacts the ammonia rich wastewater with a stripping gas of primarily air. The digestate stripper can operate at a pH that promotes the production of AB by maintaining a pH of at least 8.5 and more desirably above 9. The digestate stripper attains this required pH using only the alkalinity in the digestate and removal of the dissolved CO₂.

The digestate stripper provides a stripper gas comprising gaseous ammonia from the digestate that passes to a stripping gas scrubber. The stripping gas scrubber contacts the stripper gas with a CO₂ stream having a high mole fraction of CO₂ with the CO₂ stream typically containing CO₂ obtained from the upgrading of the biogas stream.

An AB laden stream exits the stripping gas scrubber and is cooled to a temperature that promotes the precipitation of some of the dissolved AB. Thus, depending on the desired AB “product”, a relatively dilute solution and/or a slurry containing precipitated and/or dissolved AB can be generated from the digestate by manipulating the pH of the scrubbing solution and the temperature of an AB laden stream stripped by the stripping gas. The AB laden stream exiting the stripping gas scrubber may also contain AC.

In its most basic form, the method starts with a biogas stream and a digestate liquid that both emanate from an anaerobic digester. Upgrading the biogas stream produces a methane rich stream and a CO₂ rich gas. Air stripping of the digestate liquid generates a stripper gas containing ammonia and CO₂. A scrubber scrubs the stripper gas with a scrubbing solution in a scrubber. The scrubber may contain additional CO₂ originating from the biogas upgrading process. All of the CO₂ used in the process can consist essentially of CO₂ generated during the anaerobic digestion process. Contact of the stripper gas with the CO₂ rich stream produces a liquid scrubber effluent that contains AB. The liquid scrubber solution may be chilled to reduce the solubility of AB and foster conditions for precipitation of AB particles that can then provide an AB product. The production of AB in the scrubber takes up CO₂ so that the liquid scrubbing solution is lean in CO₂. Recovery of AB particles from the liquid scrubber solution leaves a lean scrubber solution. Addition of CO₂ into the lean scrubbing solution along with optional reheating again provides the CO₂ rich solution for passage to the stripping gas scrubber. In the preferred mode of this invention rejected CO₂ from the biogas upgrading replenishes the lean the scrubbing solution with most or all of its CO₂.

In an expanded embodiment, the method recovers AB from an ammonia rich digestate liquor that also contains dissolved CO₂ produced by an anaerobic digester. The method also recovers a biogas comprising methane and carbon dioxide produced by the anaerobic digester. The method separates at least some of the biogas into a methane rich gas and a CO₂ rich gas comprising dissolved CO₂.

This method of this expanded embodiment then strips at least some of the digestate liquor with an air stream in a digestate stripper while attaining a pH in the digestate stripper of at least 8.5 and preferably above 9 in the digestate stripper. The digestate stripper produces an ammonia lean solution that leaves the digestate stripper as a rejected stream and produces the stripper gas comprising ammonia and CO₂.

The method of this expanded embodiment continues with some or all of the stripper gas passing to the stripping gas scrubber where the stripper gas contacts the CO₂ rich scrubbing solution. A resulting liquid scrubber effluent from the CO₂ scrubber comprising AB and dissolved CO₂ is cooled to produce a cooled scrubber effluent. The cooled scrubber effluent has a temperature low enough to at least promote precipitation of AB and preferably cooled to a temperature that initiates formation of AB particles. AB particles, and if present AC particles, are withdrawn from the cooled scrubber effluent as a precipitate containing slurry.

The remainder of the cooled scrubber effluent after withdrawal of the precipitate containing slurry leaves a lean scrubbing solution that has a lower concentration of CO₂ than the CO₂ rich scrubbing solution. The lean scrubbing solution passes next through a heater that heats all or a portion of the lean scrubbing solution to provide a heated scrubbing solution that is contacted with at least some of the CO₂ rich gas in a CO₂ saturator thereby producing the CO₂ rich solution.

A make-up stream comprising water, preferably fresh water, is added to the lean scrubbing solution and/or the heated scrubbing solution at a location or locations upstream of CO₂ saturator to provide the CO₂ rich gas. An AB product is recovered from the precipitate containing slurry. This may be used as is or further concentrated via any number of well-known processes.

The method can process common waste streams such as agricultural, municipal and/or industrial wastewater. It can also process a wide variety of other feeds including food wastes, whey or biomass byproducts from the production of biofuels. When solids are present, the digester typically takes all the solids.

This invention finds significant use in relation to animal husbandry. When used in this application the invention sequesters a significant fraction of the ammonia (and some of the CO₂) generated via anaerobic digestion of animal manure while also generating a methane stream suitable for providing a renewable natural gas product. Since feed material comprises manure, the method in the case produces products from renewable sources and provides both green ammonia compounds and green methane as products. In this case the invention satisfies the objective of providing such products in a simple, cost-effective manner that is readily implemented in animal farming operations such as dairy, hog or poultry farms.

Accordingly in an embodiment of the invention, particularly suited for farm animal operations and recovery of AB from manure, the method uses the steps as described in the previous embodiment and incorporates additional integrated steps to recover AB from a raw waste stream comprising water and suspended solids. The initial step separates the raw waste stream to provide a stream with a high amount of suspended solids and a digester feed stream. The suspended solids stream passes to an acidification reactor that hydrolyzes the solids and produces primarily volatile fatty acids. In addition, the acidification reactor discharges recovered water that can serve as a water make-up stream as described in the previous embodiment. An anaerobic digester receives at least a portion of the digester feed and therein produces a digestate liquor containing ammonia and a biogas stream. Animal manure has a very high alkalinity which after stripping of the CO₂ readily enables the digestate to achieve a pH of at least 8.5 and preferably higher. The biogas undergoes further separation to produce a methane rich stream, ultimately saleable as RNG, and to produce a CO₂ rich stream that saturates the heated scrubbing solution with CO₂ to supply the CO₂ rich scrubbing solution as previously described.

Thus, the invention provides a multiplicity of environmental advantages. The elimination of any chemical stripping provides a source a green AB and may also provide additional green ammonia or other green compounds. The invention can also split the biogas to effect two environmental benefits. With additional purification as may be required, the resulting methane rich stream delivers RNG to deliver one benefit. In addition, conversion of ammonia by contact with the CO₂ in the biogas can provide another benefit by sequestering up to 10% or more of the CO₂ mass that can emanate from many biomass and farming operations.

### BRIEF DESCRIPTION OF THE DRAWINGS

FIG. 1 is a flow diagram for the practice of the invention showing the addition of a precipitation reactor and a CO₂ separator.

FIG. 2 is a flow diagram for the practice of this invention showing a full flow arrangement for starting with a water waste stream and producing an AB product and a methane rich gas.

### DETAILED DESCRIPTION OF THE INVENTION

This invention can recover AB from any aqueous stream that contains large amounts of ammonia or ammonium. Most streams that are used in the invention will have recoverable ammonia or ammonium of less than 1.0 wt. %, less than about 0.5 wt. % and more typically in a range of 0.1 to 0.2 wt. %.

A more complete description of the invention is given in conjunction with the following explanation of the Figures. For clarity the figures omit additional process equipment that is well known to those skilled in the art and readily incorporated without explanation by those skilled in the art. Such equipment includes pumps, compressors, temperature sensors, pressure sensors, control loops and other such items.

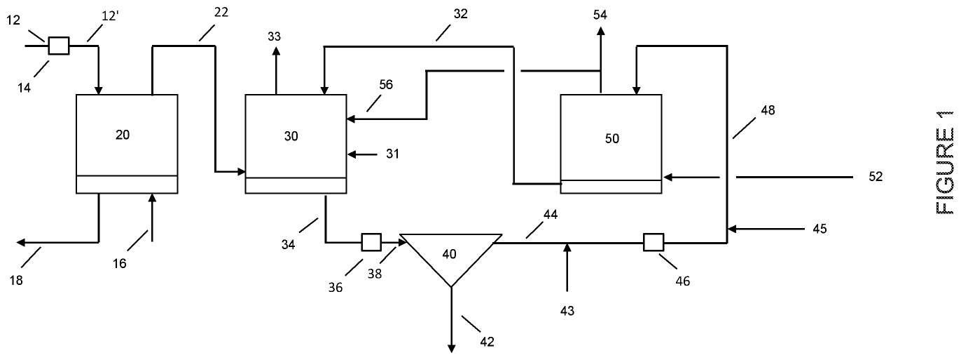

FIG. 1 shows a block diagram of the basic embodiment of the invention. The primary vessels of the method include a digestate stripper 20, a stripping gas scrubber 30, a precipitation vessel 40 and a CO₂ saturator 50.

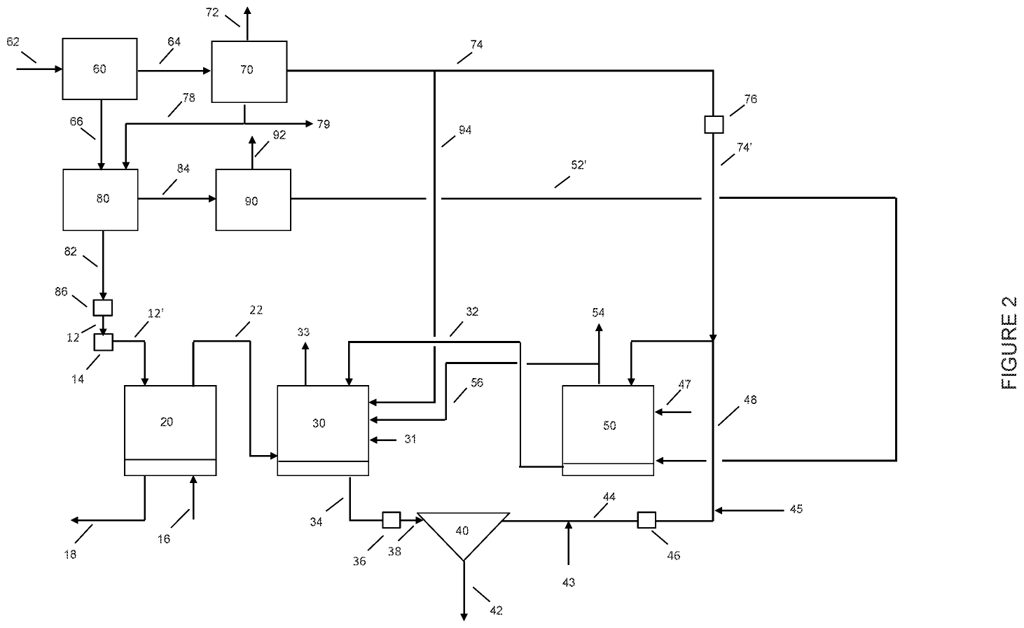

Stripper 20 receives a digestate stream 12 comprising a digestate liquor from an anaerobic digester (as shown in FIG. 2) that primarily contains dissolved ammonia and CO₂. The digestate stream 12 may also contain lesser amounts of residual solids, dissolved salts and organics. Depending on the remaining particulate material in digestate stream 20, trace suspended solid treatment (not shown) may provide a benefit to the operation of the scrubber The digest stream may optionally receive heat from an optional heating zone 14 that heats the digestate stream to an input temperature suitable for the desired operation of the digestate stripper 20. Any conventional heater or heat exchanger can provide the desired heating in zone 14. The arrangement of FIG. 1 shows an optionally heated digestate stream 12′ entering digestate stripper 20.

Digestate stripper 20 strips ammonia from the digestate by contact therein with a stripping gas 16. The stripping gas will preferably consist essentially of air but also may include minor amounts of H2S. The pressure in digestate stripper 20 is normally at or near atmospheric pressure but in certain cases can be operated under a vacuum that will usually range from 0.5 to 0.9 atmospheres.

Contact between the digestate and the stripping gas 16 in the digestate stripper will cause the pH in the digestate stripper to attain a value of at least 8.5 and preferably more than 9. The stripper needs no chemical addition to control the pH in the digestate stripper. Preferably the digestate stripper operates without any chemical addition.

Stripping of dissolved CO₂ in the digestate stripper along with the alkalinity associated with a high amount of bicarbonate and carbonate in the digestate was found to naturally raise the pH in digestate stripper 20 to a pH of 8.5 to 9 or about so that the digestate stripper requires no pH adjustment and can operate without the use of any additives. Temperature in the stripping vessel can range from room temperature to 50° C. or thereabout. Even at about room temperature the natural rise in the pH can enable a recover on the order of 50 to 70% of the ammonia in the digestate liquor.

An ammonia lean solution 18 exits a lower portion of digestate stripper 20 and a stripper gas 22 passes out of an upper portion of digestate stripper 20. The ammonia lean solution typically comprises a waste stream since it usually contains some solid material as well as other chemicals in trace amounts. Thus, the ammonia lean solution 18 may undergo waste treatment before disposal.

Stripper gas stream 22 will principally comprise gaseous ammonia along with water vapor, CO₂ and other constituents typically present in trace amounts. Such trace constituents typically include sulfur compounds. Optionally (and not shown), a dilute aqueous ammonia solution could be generated by separating the CO₂ from the ammonia in stripper gas 22 and scrubbing/condensing the ammonia from that CO₂ depleted gas stream. An optional pressure differential control between the digestate stripper 20 and scrubber 30 can include placement of a pressure control valve (not shown) in the conduit for transferring stripper gas 22.

Stripping gas scrubber 30 receives stripper gas 22 and contacts the stripping gas stream with a source of CO₂. The stripper gas contact with CO₂ converts the gaseous ammonia to AB and as well as some AC. Absorption of the gaseous ammonia into the scrubbing solution converts it to predominantly a dissolved ammonium form. The presence of the CO₂ produces a high concentration of AB in scrubber 30. In most cases the scrubber 30 as well as the CO₂ rich scrubbing gas has a neutral or near neutral pH. The scrubbing vessel temperature ranges on the low side from 5° C. to 10° C. higher than the stripping vessel can go up to a temperature of 50° C. or thereabout.

A CO₂ rich scrubbing solution 32 (as hereinafter described) enters scrubber 30 (in most cases an upper portion of scrubber 30) and supplies all of the necessary CO₂ to substantially convert the gaseous ammonia to the AB and at times some AC. Alternatively or in addition to the CO₂ scrubbing solution 32, CO₂ may optionally enter scrubber 30 directly via an optional CO₂ input stream 31. The CO₂ input stream 31 can feed CO₂ from any available CO₂ source. In the case of manure management, the CO₂ sources include, without limitation, CO₂ from the biogas upgrade system and CO₂ from an acid phase digestion system as long as it is generated within a manure management process. With regard to CO₂ entering scrubber 50, unless an external source provides CO₂ via optional input stream 31, all CO₂ that enters saturator 30 is derived from the digester that produces the digestate. An ammonia depleted stripper gas stream 33 exits scrubber 30 and if necessary subject to further treatment before release.

Scrubber 30 produces a liquid scrubber effluent 34 that comprises AB and water and may include other ammonia derived compounds such as AC as well as trace chemicals such as the previously mentioned sulfur compounds. The AB concentration in liquid scrubber effluent 34 may range from 0.8% to 14% wt. % depending on the CO₂ concentration in scrubber 30 as well as the solubility resulting from the temperature liquid in the scrubber.

To obtain the desired AB product, a relatively dilute solution and/or a precipitate can be generated by manipulating the temperate and pH of the scrubbing solution. Liquid scrubber solution effluent 34 typically passes to a cooler often in the form of a heat exchanger 36. Chilling of effluent 34 reduces the solubility of the AB and increases the mass of AB particles for removal from solution 34. Preferably the liquid scrubber effluent is cooled to a temperature of 20° C. to 25° C. Chilling may take place in any number of ways. For example, by a separate chilling step in a stand-alone chiller/heat exchanger. This invention particularly benefits from passing the liquid scrubber effluent 34 to chiller 36 and then passing a cooled scrubber effluent to a precipitation reactor 40 via a line 38.

Precipitation reactor 40 generates a precipitate containing slurry 42 and a lean scrubbing solution 44. Precipitate containing slurry 42 will comprise primarily AB. Precipitation reactor 40 operates at conditions to increase the amount precipitate in slurry 42.

Slurry 42 typically can be recovered as a final product or can be sent to further processing/concentrating as desired. Such processing typically can include physical separation via membrane separation, centrifugation, and vortex separators to mention a few that anyone trained in the art can envision.

Lean scrubbing solution 44 passes to a heating zone 46 to heat the lean scrubbing solution and provide a heated scrubbing solution 48 that enters a CO₂ saturator 50. Heating zone 46 raises the temperature of lean scrubbing solution 44 to a range of from 35° C. to 50° C. and preferably from 40° C. to 50° C. Any conventional heater or heat exchanger can provide the desired heating in zone 46. A pump or pumps (not shown) can transport and adjust the pressure of lean scrubbing solution 44 and heated scrubbing solution 46 in its delivery to CO₂ scrubber 50 via line 48.

Water, preferably fresh water, is added back to the scrubbing solution to make up for the water lost to precipitate containing slurry 42. Water is also lost by the presence of water in the CO₂ off gas, in the scrubber off gas and/or in the liquid scrubber effluent> The water may be added at any location that such that it eventually reaches CO₂ saturator 50. For example, the CO₂ saturator may receive water by passing it into lean scrubbing solution 44, heated scrubbing solution 48 and directly into CO₂ saturator 50, or any combination thereof. FIG. 1 shows these possible water entry locations as steams 43, 45, and 47, respectively.

Saturator 50 saturates the heated scrubbing solution with CO₂ recovered from biogas produced by the anaerobic digestor that provides digestate stream 12 (shown in FIG. 2). This recovered CO₂ passes to saturator 50 as a CO₂ rich gas 52. Saturator 50 usually operates at or about ambient pressure. Saturator 50 produces the CO₂ rich solution 32 and a CO₂ off gas stream 54. The CO₂ rich gas 52 typically has a CO₂ concentration ranging from 90 to a 99% mole fraction. Off gas stream 54 can optionally provide an additional source of CO₂ for scrubber 30. Thus, a CO₂ stream 56 containing CO₂ recovered from off gas stream 54 may enter scrubber 30 either with a stripper gas (not shown) or pass directly into scrubber 30.

FIG. 2 shows an embodiment of the invention that provides additional advantages, benefits, and products. In this embodiment the method starts with a raw waste stream as the feed. In FIG. 2 the reference numbers are the same for like elements as those shown in FIG. 1. The following description only describes the added method steps that don't appear in FIG. 1; that were not described in conjunction with the description of FIG. 1; and any method operations that vary between the method as described in FIG. 1 versus that described in FIG. 2.

The initial feed comprises a raw waste stream 62 that contains a high amount of solid material. In farm operations that rely on animals, raw waste stream 62 can comprise manure and other semi-solid waste and/or waste milk. Such streams are typically sourced from holding tanks or settling ponds. Accordingly, a raw waste stream 62 passes into a solids removal unit 60 that concentrates the removed solids into a suspended solids stream 64 and a wastewater steam 66. Wastewater stream 66 has a low enough concentration of total suspended solids to allow one or more moderate to high rate anaerobic treatment systems to be used including but not limited to anaerobic filters, anaerobic contact processes and high rate granular systems such as an upflow anaerobic sludge blanket, internal circulation reactor, expanded granular sludge blanket, external circulation sludge blanket, and similar processes. Any type of solids removal unit may be used that efficiently provide a high degree of suspended solids removal from raw waste stream 62. Common equipment for the separation of such solids include centrifugation, membrane filtration and/or simple screening or filtration.

A suspended solids stream 64 passes suspended solids from solids removal unit 60 to an acidification reactor 70. Before entering or as part of an acidification reactor pretreatment, the solids in stream 64 may undergo treatment to enhance degradability via a variety of different mechanical or organically acceptable methods (not shown). These methods include grinding to reduce particle size, steam explosion, enzymatic hydrolysis and high temperature treatment.

Acidification reactor 70 will preferably operate with a short hydraulic retention time on the order of 0.5 to 2 days. Typically, acidification reactor 70 operates at ambient pressure in a temperature range of from ambient to 35° C. Acidification reactor 70 hydrolyzes the solids and ultimately converts them to soluble products, primarily volatile fatty acids (acetic, propionic, butyric and higher order acids) recovered via line 74. Gases produced in acidification reactor 70 exit via line 72 to be processed as required.

Acidification reactor 70 provides a recovered water stream 74 that supplies at least a portion of the make-up water to replace water lost with removal of precipitate containing slurry 42 as previously described. Make-up water from the acidification reactor can contain volatile organic and/or inorganic acids that will promote ammonia capture. Preferably, the volatile fatty acids are derived from organic sources. Such organic sources may include animal waste.

The recovered water 74 may enter CO₂ saturator 50 via addition to heated scrubbing solution 48 as depicted in FIG. 2; by direct introduction into saturator 50; or by addition other streams entering saturator 50. Stream 74 may pass through a filter 76 to remove any residual solids and a stream 74′ with reduced residual solids exits filet 76. Recovered water may as an alternative or in addition thereto, pass ultimately to the CO₂ rich solution at the location of water addition streams 43, 45 and/or 47.

The recovered water stream 74 provides water that ultimately enters the CO₂ rich scrubber solution 32. The addition of this water improves ammonia capture in stripping gas scrubber 30 by capturing ammonia as organic acid-ammonia complexes such as ammonium acetate, and ammonium propionate for example. The improved ammonia capture results in higher concentrations of ammonium in liquid scrubber effluent 34 so that the resultant precipitate containing slurry 42 contains increased ammonia concentrations.

The solids processed in acidification reactor 70 can optionally receive high energy shearing via pumps or other means well known and understood, to significantly reduce the size of the solids to maximize the amount of hydrolysis that can occur. In this case, optionally, an acidification effluent 78 comprising mostly solids and water can pass to an anaerobic digester 80. Alternately, the effluent stream from acidification reactor 70 can be wasted via line 79. As its primary feed, anaerobic digester 80 receives wastewater stream 66 from suspended solids removal unit 60. Digester 80 produces two primary outputs; one is a biogas stream 84. The other output is digestate liquor stream 82 that that provides the essential input of digestate liquor stream 12 as described in conjunction with FIG. 1 Digestate liquor stream 82 may undergo optional filtration of fine particles in a filter 86 before it passes as digestate liquor stream 12 for processing as described in conjunction with FIG. 1.

Biogas stream 84 passes to a biogas purification section 90 that separates the biogas into a CO₂ rich gas 52′ and a methane steam 92. CO₂ rich gas 52′usually provides CO₂ for saturator 50. Line 94 may also carry at least a portion of CO₂ rich gas 52 to scrubber 30 via CO₂ input stream 31. Line 94 may supply all the CO₂ needed in scrubber 30 to produce the AB in liquid scrubber effluent 34.

Purification section 90 typically provides a high purity methane in methane stream 92. Thus, stream 92 can provide, with any additionally needed purification, an NRG product stream.

The pressure of purification section 92 is preferably sufficient for the operation of saturator 50. Because a similar pressure can be used in both purification section 92 and saturator 50, the need for any significant pressure adjustment between purification section 92 and saturator 50 is eliminated.

As described, the present invention provides numerous advantages, some of which have been described above and others which are inherent in the invention. Also, modifications may be proposed without departing from the teachings herein. Accordingly, the scope of the invention is only to be limited as necessitated by the accompanying claims.

I claim:

1. A method for recovering ammonium bicarbonate from an ammonia rich digestate liquor containing dissolved CO₂ and a biogas comprising methane and carbon dioxide produced by an anaerobic digester, the method comprising:
a.) separating at least a portion of the biogas into a methane rich gas and a CO₂ rich gas;
b.) stripping at least a portion of the digestate liquor in a digestate stripper with a stripping gas comprising air while maintaining at least a portion of the digestate stripper at a pH of at least 8.5 wherein the pH level is achieved at least in part by the stripping of dissolved CO₂ in the digestate and the stripper produces an ammonia lean solution that passes out of the digestate stripper and produces a stripper gas stream comprising ammonia and CO₂;
c.) passing at least a portion of the stripper gas stream to a stripping gas scrubber and contacting the stripping gas stream with a CO₂ rich scrubbing solution comprising dissolved CO₂;
d.) recovering a liquid scrubber effluent from the stripping gas scrubber wherein the liquid scrubber effluent comprises ammonium bicarbonate and a dissolved CO₂;
e.) cooling at least a portion of the liquid scrubber effluent to at least initiate precipitation of ammonium bicarbonate from the liquid scrubber effluent and produce a cooled scrubber effluent;
f.) passing the cooled liquid scrubber effluent to a precipitation reactor that precipitates at least a portion of the ammonium bicarbonate from the liquid scrubber effluent to produce a precipitate containing slurry comprising ammonium bicarbonate and to produce a lean scrubbing solution having a lower concentration of CO₂ than the CO₂ rich scrubbing solution;
g.) heating a least a portion of the lean scrubbing solution to produce a heated scrubbing solution and contacting the heated scrubbing solution with at least a portion of the CO₂ rich gas to produce the CO₂ rich scrubbing solution;
h.) adding a make-up stream comprising water to at least one of the lean scrubbing solution, the heated scrubbing solution, the CO₂ rich solution, and/or directly into a CO₂ saturation vessel; and,
i.) recovering ammonia bicarbonate from the precipitate slurry.

2. The method of claim 1 wherein an ammonia bicarbonate product is produced from the recovered ammonium bicarbonate and a renewable natural gas is produced from the methane rich gas.

3. The method of claim 1 wherein the make-up stream contains volatile organic and/or inorganic acids to promote ammonia capture.

4. The method of claim 3 wherein the make-up stream contains volatile fatty acids derived from organic sources.

5. The method of claim 4 wherein the organic sources comprise animal waste.

6. The method of claim 1 wherein the CO₂ rich scrubbing solution contains at least a portion of the CO₂ rich gas and is derived, at least in part, from the heated scrubbing solution.

7. The method of claim 1 wherein digestate liquid enters the digestate stripper at a temperature no greater than 50° C.

8. The method of claim 1 wherein the precipitate containing slurry additionally comprises ammonium carbonate, ammonium, and ammonium organic acid complexes.

9. The method of claim 1 wherein a precipitation reactor precipitates at least a portion of the ammonium bicarbonate from the liquid scrubber effluent.

10. The method of claim 1 wherein an anaerobic digester converts a biomass material containing ammonia and organic nitrogen containing compounds to produce the digestate liquor and the biogas.

11. The method of claim 10 wherein the waste material comprises agricultural waste.

12. The method of claim 2 wherein the stripping gas stream consists essentially of air and water vapor.

13. The method of claim 1 wherein the liquid scrubber effluent contains acidified ammonium.

14. The method of claim 1 wherein the digestate liquor contains dissolved CO₂ that when stripped from solution raises the pH of the digestate stripper.

15. A method for recovering ammonium bicarbonate from a wastewater stream comprising water and suspended solids, the method comprising:
a.) separating at least a portion of the suspend solids from the wastewater to produce a suspended solids stream and an ammonia rich stream;
b.) passing at least a portion of the ammonia rich stream to an anaerobic digester to produce a biogas comprising methane and CO₂ and to produce a digestate liquor comprising additional ammonia;
c.) separating at least a portion of the biogas into a methane rich stream and a CO₂ rich gas;
d.) stripping the digestate liquor in a digestate stripper by contact with a stripping gas comprising air and water vapor wherein at least a portion of the digestate stripper retains liquid therein at a pH of at least 8.5;
wherein the digestate stripper produces an ammonia lean solution and a stripper gas stream containing ammonia;
e.) passing at least a portion of the stripper gas stream to a stripping gas scrubber and contacting the stripping gas stream with a CO₂ rich scrubbing solution;
f.) recovering a liquid scrubber effluent from the stripping gas scrubber, the liquid scrubber effluent comprising ammonium bicarbonate and ammonium carbonate wherein the liquid scrubber effluent has a low concentration of dissolved CO₂ relative to the CO₂ rich solution.
g.) cooling at least a portion of the liquid scrubber effluent to produce a cooled scrubber effluent and passing the cooled scrubber effluent to a precipitation reactor that precipitates at least a portion of the ammonium bicarbonate and ammonium carbonate from the liquid scrubber effluent to produce a precipitated slurry containing ammonium bicarbonate and ammonium carbonate and to produce a lean scrubbing solution having a lower concentration of ammonium bicarbonate than the feed to the precipitation reactor.
h.) heating at least a portion of the lean scrubbing solution to produce a heated scrubbing solution;
i.) passing the heated scrubbing solution and the CO₂ rich gas to a CO₂ saturator to produce the CO₂ rich scrubbing solution and discharging a CO₂ off gas from the CO₂ saturator;
j.) adding a make-up stream comprising water to at least one of the heated scrubbing solution or the CO₂ saturator;
k.) recovering ammonium bicarbonate and ammonium carbonate from the precipitated slurry and, recovering the methane rich stream for production of a refined natural gas

16. The method of claim 15 wherein at least a portion of the suspended solid stream is passed to an acidification reactor that hydrolyzes the suspended solids to produce volatile fatty acids and at least a portion of the volatile fatty acids are introduced into the stripping gas scrubber;

17. The method of claim 16 wherein the volatile fatty acids are introduced into the stripping gas scrubber by mixing at least a portion of the volatile fatty acids with the CO₂ rich scrubbing solution.

18. The method of claim 15 wherein the wastewater comprises waste effluent from farming operations.

19. The method of claim 16 wherein the biogas contains H2S; wherein the biogas is treated to oxidize the H2S and to produce a liquid oxidation stream rich in sulfuric acid and/or dissolved sulfates; and wherein the make-up stream comprises at least a portion of the liquid oxidation stream.

20. The method of claim 15 wherein at least a portion of the CO₂ off gas is passed to the stripping gas scrubber.

21. A method for recovering ammonium bicarbonate from a wastewater stream comprising water and suspended solids, the method comprising;
a.) separating at least a portion of the suspend solids from the wastewater to produce a suspended solids stream and an anaerobic feed stream;
b.) passing the anaerobic feed stream to an anaerobic digester to produce biogas comprising methane and CO₂ and to produce a digestate liquor comprising ammonia;
c.) separating at least a portion of the biogas into a methane rich stream and a CO₂ rich gas;
d.) stripping at least a portion of the digestate liquor in a digestate stripper by contact with a stripping gas comprising air and water vapor in the digestate stripper at a temperature of from 35 to 50° C. wherein the digestate stripper retains liquids therein at a pH of at least 8.5; wherein stripping the dissolved CO₂ from the digestate liquor raises the pH of the digestate stripper; and wherein the digestate stripper produces an ammonia lean solution and a stripper gas stream containing ammonia;
e.) passing at least a portion of the stripper gas stream to a stripping gas scrubber and contacting the stripping gas stream with a CO₂ rich scrubbing solution;
f.) passing at least a portion of the suspended solids stream to an acidification reactor that hydrolyzes the suspended solids to produce volatile fatty acids and a recovered water stream and passing at least a portion of the volatile fatty acids into the stripping gas scrubber;
g.) recovering a liquid scrubber effluent from the stripping gas scrubber, the liquid scrubber effluent comprising ammonium bicarbonate and ammonium carbonate;
h.) cooling at least a portion of the liquid scrubber effluent to produce a cooled scrubber effluent;
i.) passing the cooled scrubber effluent to a precipitation reactor that precipitates at least a portion of the ammonium bicarbonate. ammonium carbonate and ammonium organic acid compounds to produce a lean scrubbing solution having a lower concentration of CO₂ than the CO₂ rich scrubbing solution;
j.) heating at least a portion of the lean scrubbing solution to produce a heated scrubbing solution and passing the heated scrubbing solution and the CO₂ rich gas to a CO₂ saturator to produce the CO₂ rich scrubbing solution and discharging a CO₂ off gas from the CO₂ saturator;
k.) adding a make-up stream comprising water to at least one of the heated scrubbing solution or the CO₂ saturator wherein the make-up stream comprises at least a portion of recovered water stream;
l.) recovering ammonium bicarbonate and ammonium carbonate from the precipitated slurry; recovering the methane rich stream for production of a refined natural gas; and recovering volatile fatty acids from the acidification reactor.

22. The method of claim 21 wherein the acidification reactor provides a solids and water to the anaerobic digester.
